(19) United States
(12) Patent Application Publication
(10) Pub. No.: US 2014/0031768 A1
(43) Pub. Date: Jan. 30, 2014

(54) LIQUID DISPENSING CONTAINER

(71) Applicant: Shunsuke Takaki, Sagamihara-city (JP)

(72) Inventor: Shunsuke Takaki, Sagamihara-city (JP)

(21) Appl. No.: 14/110,451

(22) PCT Filed: Apr. 4, 2012
(86) PCT No.: PCT/US12/32052

§ 371 Date: Oct. 8, 2013

(30) Foreign Application Priority Data
Apr. 22, 2011 (JP) 2011-096064

(51) Int. Cl. A61F 5/44; A61M 35/00

(52) U.S. Cl. CPC A61F 5/4404; A61M 35/003
USPC 604/290; 604/310

(57) ABSTRACT

Disclosed is a container capable of dispensing a liquid contained in the container by drops while keeping the liquid in an uncontaminated state. Also disclosed is a method of dispensing a liquid by drops using the container, and a method of removing a stoma pouch flange using the container. The liquid storing container comprises a storage portion with a sealed periphery and a nozzle portion connected with the storage portion for dispensing a liquid contained in the storage portion by drops to the outside. The container has an enclosed inner space. Liquid is dispensed by drops by peeling apart a sealed nozzle tip portion that is oriented toward the tip of the nozzle portion to form a drop dispensing opening.

### BACKGROUND

1. Technical Field

The present invention relates to a liquid dispensing container. The present invention further relates to a method of dispensing a liquid by drops using the container and a method of removing a stoma pouch flange using the container.

2. Related Art

To illustrate, on a person requiring a colostomy or urostomy, a stoma is formed causing a section of the digestive tract or urinary tract to protrude from the body, and a stoma pouch constituted by a donut-shaped flange and a vinyl bag, or the like, is attached. The stoma pouch is attached by adhering the donut-shaped flange surrounding the stoma to the skin, and must be replaced one or more times each day or number of days. When removing the flange from the skin during replacement, a remover is infiltrated between the skin and the flange in order to prevent skin injury or skin irritation, and an adhesive strength is weakened whereby removal can be accomplished.

At this point, if the remover becomes contaminated, there is a risk of the stoma becoming contaminated, leading to inflammation or infection; thus, it is necessary to use a remover that has been maintained in an uncontaminated state.

Remover products include liquid types contained in multiple-dose bottles, and napkin or liquid types individually packaged as single doses. In the case where the stoma bag replacement operation is performed by the user in question, the remover is usually applied with one hand. At this time, with a liquid type contained in a bottle for multiple doses, a front-end portion of the bottle may become unhygienic through repeated use, and there may be an increased risk of infection. In the case when using a product of a napkin type for a single dose, there is still a hygienic problem because the napkin is applied by hand, and remover may furthermore be deposited on the hand and become sticky, thereby becoming an impediment to subsequent operation. Additionally, in the case where a liquid type for a single dose is used, remover may fly out as soon as an individual package is opened and contaminate clothing, and the like, or it may not be possible to apply remover to the area where removal is to be performed.

To illustrate, an ophthalmic liquid preparation dispensing system, including a package containing a pad having absorbed an ophthalmic liquid preparation, is described in Japanese Translation of Published PCT Application No. 2002-516714. With this system, a portion of the package is removed to form an opening, a portion of the pad having absorbed liquid preparation contained in the package is caused to protrude from the opening, and the ophthalmic liquid preparation is dripped from the front end portion of the protruding pad.

The remover system described in Japanese Translation of Published PCT Application No. 2002-516714 has the problem that, because portion of the pad impregnated with liquid preparation is caused to protrude out of the opening, the protruding pad contacts the outside of the package, becoming contaminated.

### SUMMARY

The disclosed container is capable of dispensing a liquid by drops while preventing the liquid from contacting the contaminated outer portion of the container, keeping the liquid in an uncontaminated state. Also disclosed is a method of dispensing a liquid by drops using the container, and a method of removing a stoma pouch flange using the disclosed container.

Specifically, the present invention provides a liquid storing container including a storage portion formed from a single body front and back of a flexible material sealed around a periphery thereof, a nozzle portion communicating with the storage portion and acting as a liquid channel for dispensing a liquid stored within the storage portion to outside the container, and an enclosed inner space being formed by a seal. The liquid storing container also includes a nozzle tip portion formed by sealing the nozzle portion toward a tip thereof, a tab portion connected to the nozzle tip portion and constituted by a front surface tab portion and a rear surface tab portion wherein the front and rear surfaces of the flexible material are not sealed, a shoulder portion disposed in a direction of the nozzle tip portion opposite to that of the tab portion, and a body portion connected to the shoulder portion; wherein an angle formed by a line extending left and right from a base portion of the nozzle tip portion formed at a boundary between the nozzle tip portion and the shoulder portion in parallel with a lower edge and a ridge line of an upper portion of a seal portion enclosing the storage portion is from 5 to 60 degrees.

The present invention also provides a method of dispensing a liquid by drops that includes the steps of peeling apart a seal of a nozzle tip portion by pulling a front surface tab portion and a rear surface tab portion of a liquid storing container of the present invention to form a drop dispensing opening (step 1); and, following step 1, holding the tab portions along a side of the liquid storing container and turning the drop dispensing opening to face downward, and then pressing the container to dispense drops of a liquid within the liquid storing container (step 2).

The present invention also provides a method of removing a colostomy or urostomy stoma pouch flange that includes the steps of peeling apart a seal of a nozzle tip portion by pulling a front surface tab portion and a rear surface tab portion of the liquid storing container, which contains a remover for removing the flange of a colostomy or urostomy stoma pouch from the skin, in opposite directions, forming a drop dispensing opening (step 3); and, following step 3, holding the tabs down along the sides of the liquid storing container and turning the drop dispensing opening downward, then pressing the container to dispense the remover by drops onto the interface between the stoma pouch flange and the skin (step 4).

The present invention enables a container and method of dispensing a liquid by drops to be provided that allows liquid within the container to be dispensed by drop upon a necessary site while maintaining the liquid in an uncontaminated state. A removing method, by which remover can be applied easily and quickly to a desired site and a flange can be removed hygienically when replacing a stoma pouch, can also be provided by the present invention.

### BRIEF DESCRIPTION OF THE DRAWINGS

FIG. 1 is a diagram illustrating one example of the container of the present invention.

FIG. 2 is a diagram illustrating one example of the container of the present invention.

FIG. 3 is a photograph illustrating one example of the remover in a container of the present invention.

FIG. 4 is a photograph illustrating one example of the container of the present invention.

FIG. 5 is a cross-sectional view along line A-A of the container shown in FIG. 1.

FIG. 6 is a view of the container of FIG. 1 when opened.

FIG. 7 is a cross-sectional view along line A-A of FIG. 6.

### DETAILED DESCRIPTION

The configuration of the liquid storing container of the present invention will be described with reference to the drawings. FIG. 1 illustrates one example of a liquid storing container according to the present invention. In a liquid storing container 1, the periphery of a single piece of flexible material having front and back surfaces is surrounded by a seal portion 12, and a storage portion 10 constituting a sealed-off space and a nozzle portion 11 communicating therewith are formed by the seal portion 12. The nozzle portion 11 acts as a liquid channel for dispensing a liquid contained within the storage portion to the outside of the container by drops.

The seal portion toward the tip of the nozzle portion forms a tip of the nozzle portion as well as a nozzle tip portion 13. Following the nozzle tip portion 13, a tab portion 14 is formed wherein the front surface and rear surface of the flexible material are not sealed. The tab portion 14 is constituted by a front surface tab portion and a rear surface tab portion, and a drop dispensing opening is formed by pulling the front surface tab portion and rear surface tab portion in opposite directions to peel apart the seal portion of the nozzle tip portion 13 to a desired position. By forming the drop dispensing opening by peeling down to a base portion 15 of the nozzle tip portion, or to a position slightly in front (approx. 1 mm) of the base portion 15, the liquid contained within the container can be dispensed by drops from the drop dispensing opening while being kept in an uncontaminated state. A marking may be provided on the surface of the container at the nozzle tip portion so that a user can easily discern the position at which liquid can be dispensed while remaining uncontaminated. While there is the possibility of the tab portion 14 being contaminated through contact with the external environment or human hands, the nozzle tip portion 13 connected to the tab portion 14 is not contaminated by virtue of being sealed, and is kept uncontaminated until a drop dispensing opening is formed.

As seen from the outside, the liquid dispensing container 1 has a shoulder portion P continuing in a direction from the nozzle tip portion 13 opposite to that of the tab portion, as well as a body portion Q.

When dispensing drops of the liquid stored in the storage portion 10 of the liquid dispensing container 1 to the outside, the front surface tab portion and rear surface tab portion are pulled so as to pull apart the seal of the nozzle tip portion 13, then respectively folded around to the front and rear surfaces of the body portion Q and held down with the fingers, allowing liquid to be dispensed by drops from the drop dispensing opening. A foam member 17 impregnated with liquid can also be stored in the liquid dispensing container 1, in which case the foam member is pressed with the fingers from the outside of the body portion Q to dispense drops of the liquid impregnating the foam.

A line L1, extending from the base portion 15 of the nozzle tip portion in parallel to the bottom side of the liquid dispensing container 1, and an outer periphery L2 of the container 1 surrounding the seal portion at the shoulder portion P, which is a ridge line of the upper portion of the seal portion surrounding the storage portion 10, form an angle θ. The angle θ is from approximately 5 to approximately 60 degrees. Having the angle θ fall within this range enables liquid within the storage portion 10 to be dispensed by drops to the outside of the container through nozzle portion 11 and out the drop dispensing opening, while avoiding contact with the outside of the container, remaining uncontaminated, when the liquid is dispensed by drops from the container.

The liquid dispensing container 1 is formed from a flexible material. A known flexible material may be used without any particular restrictions. For example, a material having gas barrier and heat seal properties can be used. Moreover, it is preferable from considerations of maintaining the quality of the liquid contained within that the material have light-blocking properties. An example of such material is a multilayer laminate film prepared by laminating a vapor-deposited layer of a metal such as aluminum or a layer including metal foil and a resin film. Examples of resin films that can be used include: polyethylene, polypropylene, and other polyolefins; polyethylene terephthalate, and other polyesters; and nylon.

When a foam member 17 impregnated with a liquid is stored in the storage portion 10, a known soft, porous material can be used for the foam member. Examples include polyurethane foam, absorbent cotton, sponge, nonwoven fabric, and the like.

The foam member is preferably impregnated with the liquid to roughly 50 to 100% of a maximum liquid retention capacity thereof. In other words, all of the liquid is impregnated in the foam member when the foam member is stored in the container. In such a state, the liquid within the container will not drip out when the container is opened to form a drop dispensing opening and turned facing downward, but will only drip out when the container is pressed. When liquid permeates the foam member at less than 50% of the maximum fluid retention capacity of the foam member, the liquid will not sufficiently drip out even when the container is pressed; and when 100% is exceeded, liquid will drip out of the nozzle just from turning the nozzle downward after opening, even when not squeezed.

The liquid dispensing container 1 can contain a variety of types of liquid, and there is no particular limitation thereupon. In particular, it is preferable to store a liquid needing to be dispensed by drops in an uncontaminated state. Examples of such liquids include eye drops, disinfectants, bactericides, and liquid removing agents. The liquid remover may be a known volatile liquid remover. A liquid removing composition containing a methylsiloxane solvent, a nonionic surfactant, and an oil solution can be given as a specific example.

The methylsiloxane solvent is preferably a volatile methylsiloxane solvent having a boiling point of 95° C. to 170° C.; and can be selected, for example, from hexamethyldisiloxane (HMDS), octamethyltrisiloxane (OMTS), hexamethylcyclotrisiloxane (HMCTS), and mixtures thereof. In particular, hexamethyldisiloxane (HMDS) is preferable. These methylsiloxane solvents are commercially available, for example, from Shin-Etsu Chemical of Japan, Dow Corning, and other silicone manufacturers. The methylsiloxane solvent can be used in a quantity of about 85 to about 99.8 wt % based on a weight of the liquid removing composition.

Examples of nonionic surfactants that can be used include sorbitan ester surfactants (ICI Span series), polyoxyethylene sorbitan ester surfactants (ICI Tween series), polyethoxylated aklyl phenols (Triton X35, X102, and the like), and polyethylene glycol fatty acid monoesters (polyethylene glycol 400 monolaurate, polyethylene glycol 400 monooleate, and the like). The surfactant is suitably added to an extent of about 0.1 to about 10 wt % based on the weight of the liquid removing composition.

The oil solution, for example, may be an oil-compatible, adhesive-dissolving oil solution having a boiling point of 170° C. or higher and being difficult to volatilize. Examples of such oil solutions include terpenes, fatty compounds, fatty esters, and certain fatty acids. Terpenes and fatty compounds are included among these as being highly effective in softening and dissolving adhesives.

A specific example of a remover containing a methylsiloxane solvent, a nonionic surfactant, and an oil solution is TP1 remover for skin from 3M Health Care. The liquid remover mentioned above is capable of removing a variety of adhesives such as acrylic adhesives, synthetic rubber-based adhesives, silicone adhesives, and many other types of adhesives.

Because the flange of a colostomy or urostomy stoma pouch is adhered to the skin using an adhesive, the flange can be removed from the skin by dispensing drops of the liquid remover stored in the liquid dispensing container of the present invention onto the interface between the flange and the skin. In this instance, the remover within the container can be dispensed by drops onto the interface between the stoma pouch flange and the skin in an uncontaminated state by forming a drop dispensing opening according to the process described above, turning the drop dispensing opening downward while holding the tab portions at the surface of the liquid dispensing container, and pressing the container to dispense drops of the remover onto the interface between the stoma pouch flange and the skin.

The liquid dispensing container 1 may be manufactured according to a known method. For example, a laminated film may be folded in half, and a foam member disposed within as necessary. Two sides are next crimped by heat sealing while leaving one side on a top open, resulting in a pouch-shaped container having one end open. Next, liquid is injected into the container through the open end, and the container is sealed by means of heat sealing or ultrasonic sealing. Die cutting is then performed to obtain a container of a desired shape. The outside of the completed container may be further sterilized by means of gamma irradiation or the like.

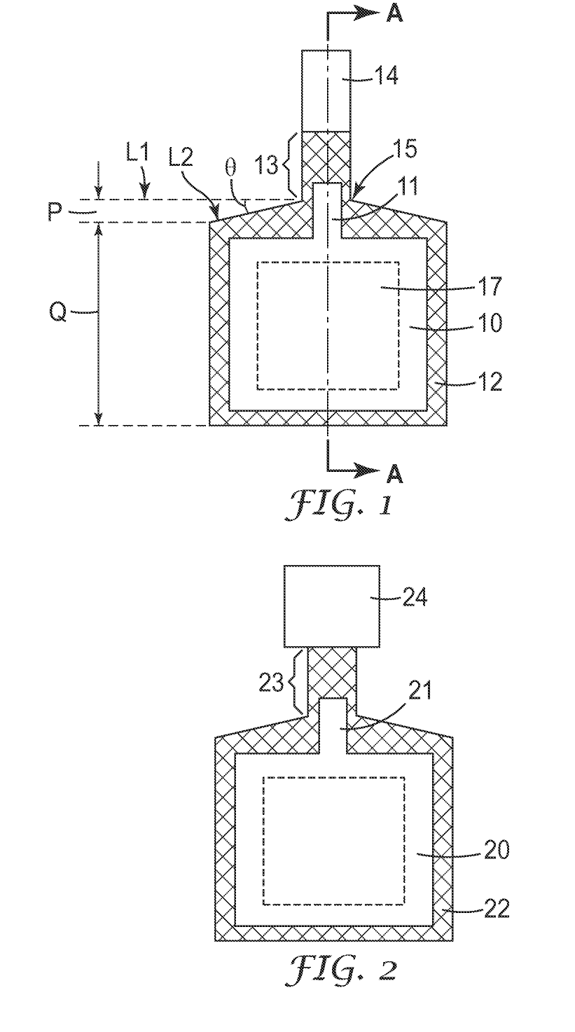

FIG. 2 illustrates one example of a liquid storing container according to the present invention. The container is almost identical to the liquid dispensing container shown in FIG. 1, but a tab portion 24 is wider than a nozzle tip portion 23. Because the tab portion 24 is wider, the front surface tab portion and rear surface tab portion are easier to grasp with the fingers and pull.

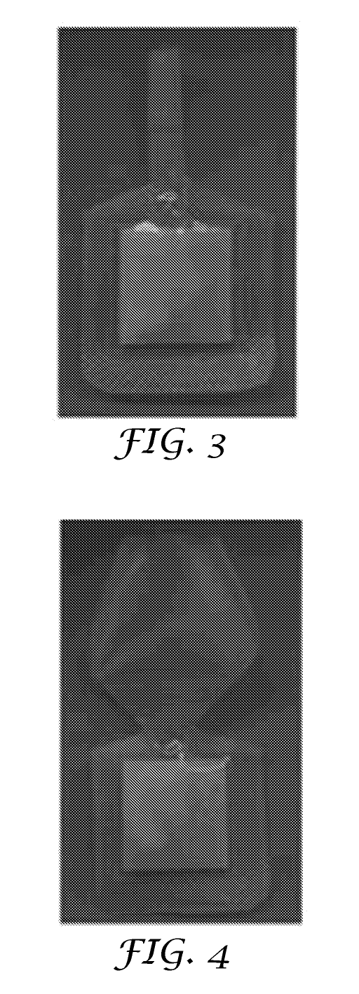

FIG. 3 is a photograph of a liquid dispensing container similar to that of FIG. 1. A liquid dispensing container 3 is constituted by a transparent material, and a storage portion contains a foam member impregnated with a liquid. The transparent material can be the resin film mentioned in the description of FIG. 1.

FIG. 4 is a photograph of one example of a liquid storing container according to the present invention. A liquid dispensing container 4 is constituted by a transparent material, similar to the liquid dispensing container 3 shown in FIG. 3, and a storage portion thereof contains a foam member impregnated with a liquid. The area from the nozzle tip portion to the tab portion is shaped like the tail of a goldfish, making the front surface tab portion and rear surface tab portion easy to grip and pull apart with the fingers.

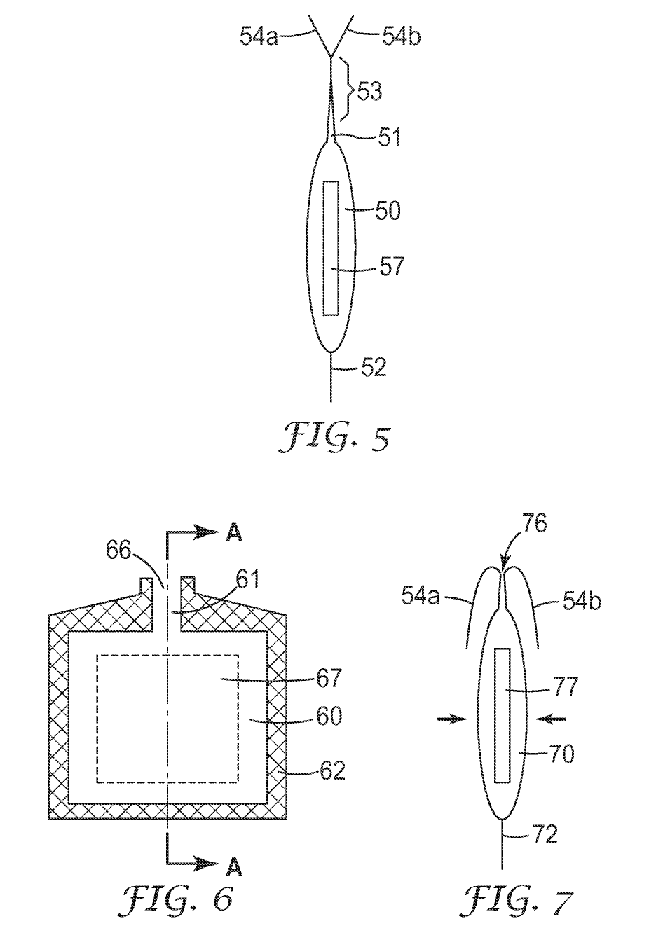

FIG. 5 is a cross-sectional view along line A-A of the liquid dispensing container shown in FIG. 1. An unsealed front surface tab portion 54a and a rear surface tab portion 54b are formed in the upper portion of the drawing, and connect to a sealed nozzle tip portion 53. A communicating nozzle portion 51 and a storage portion 50 form a space sealed off by the sealed nozzle tip portion 53 and seal portion 52. A liquid-impregnated foam member 57 is stored in the storage portion 50.

FIG. 6 shows the liquid dispensing container of FIG. 1 after having been opened. The tab portions of the liquid dispensing container 1 shown in FIG. 1 have been pulled apart to partway down the seal of the nozzle tip portion to form a drop dispensing opening 66. The tab portions and pulled-apart nozzle tip portion connected thereto are not shown in the drawing. A storage portion 60 is connected to the outside of the container through a nozzle portion 61 and the drop dispensing opening 66, and when the liquid dispensing container is turned so that the drop dispensing opening 66 faces downward, it is possible to dispense drops of liquid from the drop dispensing opening 66 without the liquid contacting the outer surface of the container.

FIG. 7 is a cross-sectional view along line A-A in FIG. 6. A drop dispensing opening 76 has been formed connected to the front surface tab portion 54a and rear surface tab portion 54b following the nozzle tip portion, which has been peeled open by pulling on the tab portions. Liquid impregnating a foam member 77 contained within a storage portion 70 passes through the nozzle portion and is dispensed by drops out of the container via the drop dispensing opening 76. By pressing upon the front surface tab portion 54a and the rear surface tab portion 54b with the fingers so as to fold along the front and rear surfaces, respectively, of the liquid storing container at this point, drops of liquid can be dispensed upon a desired site from the drop dispensing opening 76 without coming in contact with contaminated tab portions.

1-8. (canceled)

9. A liquid storing container comprising:
a storage portion formed from a single body front and back of a flexible material sealed around a periphery thereof and forming an enclosed inner space;
a nozzle portion communicating with the storage portion and acting as a liquid channel for dispensing a liquid stored within the storage portion to outside the container;
a nozzle tip portion formed by sealing the nozzle portion toward a tip thereof;
a tab portion connected to the nozzle tip portion and constituted by a front surface tab portion and a rear surface tab portion wherein the front and rear surfaces of the flexible material are not sealed;
a shoulder portion disposed in a direction of the nozzle tip portion opposite to that of the tab portion, and a body portion connected to the shoulder portion; wherein an angle formed by a line extending left and right from a base portion of the nozzle tip portion formed at a boundary between the nozzle tip portion and the shoulder portion in parallel with a lower edge and a ridge line of an upper portion of a seal portion enclosing the storage portion is from 5 to 60 degrees.

10. The liquid storing container according to claim 9, wherein the nozzle tip portion is formed with an easily peelable seal, and a drop dispensing opening is formed by pulling the front surface tab portion and the rear surface tab portion in opposite directions so as to peel apart the seal of the nozzle tip portion to a desired position.

11. The liquid storing container according to claim 9, wherein a liquid-impregnated foam member is contained within the storage portion.

12. The liquid storing container according to claim 11, wherein the liquid is a volatile liquid remover composition.

13. The liquid storing container according to claim 11, wherein the foam member is impregnated with the liquid remover composition to from 50% to 100% of a liquid retention capacity thereof

14. The liquid storing container according to claim 11, wherein the liquid is a remover for removing a colostomy or urostomy stoma pouch flange from skin.

15. A method of dispensing a liquid by drops, comprising the steps of:
peeling apart a seal of a nozzle tip portion by pulling a front surface tab portion and a rear surface tab portion of a liquid storing container according to claim 9 to form a drop dispensing opening; and
holding the tab portions along a side of the liquid storing container and turning the drop dispensing opening to face downward, and then pressing the container to dispense drops of a liquid within the liquid storing container.

16. A liquid storing container comprising:
a storage portion formed from a single body front and back of a flexible material sealed around a periphery thereof and forming an enclosed inner space;
a nozzle portion communicating with the storage portion and acting as a liquid channel for dispensing a liquid stored within the storage portion to outside the container;
a nozzle tip portion formed by sealing the nozzle portion toward a tip thereof;
a tab portion connected to the nozzle tip portion and constituted by a front surface tab portion and a rear surface tab portion wherein the front and rear surfaces of the flexible material are not sealed;
a liquid-impregnated foam member is contained within the storage portion.

17. The liquid storing container according to claim 16, wherein the nozzle tip portion is formed with an easily peelable seal, and a drop dispensing opening is formed by pulling the front surface tab portion and the rear surface tab portion in opposite directions so as to peel apart the seal of the nozzle tip portion to a desired position.

18. The liquid storing container according to claim 16, wherein the foam member is impregnated with the liquid remover composition to from 50% to 100% of a liquid retention capacity thereof

19. A method of dispensing a liquid by drops, comprising the steps of:
peeling apart a seal of a nozzle tip portion by pulling a front surface tab portion and a rear surface tab portion of a liquid storing container according to claim 16 to form a drop dispensing opening; and
holding the tab portions along a side of the liquid storing container and turning the drop dispensing opening to face downward, and then pressing the container to dispense drops of a liquid within the liquid storing container.
